(19) United States
(12) Patent Application Publication
(10) Pub. No.: US 2005/0273000 A1
(43) Pub. Date: Dec. 8, 2005

(54) Method and apparatus for providing a stimulus in magnetic resonance imaging system

(76) Inventors: William Jace Dinehart, Gainesville, FL (US); Tom Holtzworth, Gainesville, FL (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/144,868

(22) Filed: Jun. 2, 2005

Related U.S. Application Data
(60) Provisional application No. 60/576,373, filed on Jun. 2, 2004.
(60) Provisional application No. 60/630,630, filed on Nov. 24, 2004.

(51) Int. Cl. A61B 5/05

(52) U.S. Cl. 600/410

(57) ABSTRACT

The subject invention pertains to a method and apparatus for providing a video display near a MRI scanner. The subject invention can provide a visual display inside a magnet room in which a MRI scanner is located. In an embodiment, the subject invention can provide a visual display near or inside the bore of a MRI scanner so as to provide visual stimulation to a patient located in the bore of the MRI scanner. The visual display of the subject invention does not interfere with the operation of the MRI scanner and the MRI scanner does not interfere with the operation of the subject visual display. In a specific embodiment, the subject invention relates to a patient display hood that is transparent to the MRI scanner such that the magnetic fields of the MRI scanner do not significantly impact the operations of the subject PDH and such that the subject PDH produces minimal, if any, detectable RF noise in the MRI scanner.

### BACKGROUND OF INVENTION

Magnetic resonance imaging (MRI) is a popular technique for imaging a patient. Unfortunately, the MRI scanning process can require patients to lie still inside of a cylindrical shaped bore of an MRI scanner for extended periods of time. It is often desirable to provide video to patients undergoing an MRI scan. Video can also be provided to provide visual stimulation to the patient in order to allow analysis of the patient's visual and resulting cognitive processes during an MRI scan. Alternatively, video can be provided as entertainment, for example, to distract the patient during the long and tedious imaging process. However, the MRI technique utilizes large magnetic fields in and around the bore of the MRI scanner where the patient is located. The magnetic fields produced by the MRI equipment can negatively impact the operation of traditional video or other electronic equipment in or near the bore of the MRI scanner bore where the patient is located, even to the extent that the video or other electronic equipment may not function properly. In addition, the presence of traditional video or other electronic equipment in or near the bore of the MRI scanner may negatively impact the MRI equipment and/or the results of the MRI scan.

Prior techniques have been employed to provide patients in or near an MRI scanner with video and/or audio or place other electronic apparatus with a magnet room of an MRI systems. U.S. patents related to such teachings include, for example, U.S. Pat. Nos. 5,412,419; 5,864,331; 5,861,865; 5,432,544; 5,877,732; 5,627,902 and 5,339,813.

Referring to FIG. 1, a patient is shown in position within the bore of an MRI scanner. Referring to FIG. 2A, a schematic illustration of a MRI system, having a MRI scanner located in a magnet room 5 and a control room 3 adjacent to the magnet room 5, utilizing a prior art system to provide video to a patient is shown. This system incorporates a video display hood 25 that incorporates a patient microphone and various electronics for patient headphones; two turtle shaped button response units (BRUs) 27, one each for the left and right hands; a peripheral interface box (PIB) 9; and a wall mounted power supply 11. The power supply 11 provides electricity to the PIB 9 that drives the components in the magnet room 5, providing both electrical power and electrical signals through one or more bundles of copper cable. The PIB 9 also includes an RF antenna and associated circuitry to detect MRI scanner RF activity. The PIB 9 connects the magnet room 5 components to a console 13 located in a control room 3, which is adjacent to the magnet room 5, through a fiber optic cable 7. Video and audio signals are received by the PIB 9 through the fiber optic cable 7, and are decoded and converted to video and audio electrical signal components. Responses from the BRUs 27 and RF activity are collected by the PIB 9 and encoded into optical signals that are sent to the console 13 in the control room 3. The video display hood 25 is designed to be utilized inside the scanner bore and operated without interference from the MRI scanner during operations and without interfering with the MRI scanner during operations. The PIB 9 is designed for operations outside the MRI scanner. During normal operations, the PIB 9 sits on a MRI safe equipment storage cart, usually located by the bed of the MRI scanner. It can be inconvenient for the operator to have to move the PIB 9 around and have to maintain the proximity between the PIB 9 and video display hood 25 when preparing for and imaging a patient.

Alternative designs that are used in systems for providing video to patients in or near an MRI scanner include: projector based systems and goggle based systems.

Projector based systems are widely used. As an example, BrainLogics™ sells a projector for use with MRI. In a projector-based system, an LCD projector is used to generate the video display inside the magnet room. The projector may be installed inside the magnet room or it may be installed outside and projected through a hole in the magnet room Faraday cage. The projector creates the image on a screen that is seen by the patient via a mirror located on top of the patient's head. The patient's visual field is somewhat limited by the size of the mirror, by the size of the bore of the magnet and, occasionally, by the size of the patient's body. Thus, visual field coverage is somewhat limited, and, in some installations, it may vary between patients, depending on the size of the patient's body. Usually, modern LCD projectors have very high resolution (1600×1200 pixels are now quite common). However, because of the limited size of the scanner bore, they have to create a relatively small image on the screen. This is usually achieved with custom optics that often induces optical artifacts in the images. A common artifact is due to diffraction of light that causes colored rings at the borders of the screen. Thus, the image quality tends to suffer and the image colors tend to be non-uniform. Custom optics are sometimes used to correct these problems, but this requires additional costs that are sometimes larger than the cost of the LCD projector. The poor focus of the systems presents another problem associated with image quality. Because of the requirement to generate a very small picture on the screen, the focal length of the projector is quite narrow, and the screen must be positioned at the perfect position in space, to be in focus. This position may change slightly during the first twenty minutes to half an hour of operations of the system, and is attributed to the warming of the various components. As a result, the focus on the screen is often sub-optimal and it is often reached after several minutes of trial and error (sometimes this procedure can take approximately 20 minutes). Typically, projector-based systems are among the least expensive solutions, as they are easy to design and do not require any custom electronics. A major disadvantage with respect to projector-based systems is that the electronics of the projector tends to be quite noisy and requires careful shielding. The shielding of a projector can also be complicated by the need to maintain good airflow in order to keep the lamp cool. Goggle-based systems are sold for fMRI products. As examples, Resonance Technologies sells LCD based goggles and Avotec sells fiber-optic based goggles.

LCD based goggles have two small LCD screens that sit above the patient's eyes, like a pair of slightly oversized glasses. The LCD panels and their electronics sit very close to the patient's eyes. Typically, the LCD resolution can be up to 1024×768 pixels per panel. The LCD goggles offer the possibility of stereo 3D vision. The LCD goggles can be combined with ceramic headphones, a patient microphone, and patient response devices. The goggles sit on the patient's face inside the head coil. High-resolution head coils, such as the high-resolution head coil (HRH head coil) by MRI Devices Corporation, can have a rather tight fit around the head and the face of the patient, which may not leave enough room for the typical goggles. The goggle system is designed with the video electronics placed right above the eyes of the patients. This design may have an impact on the SNR of the system and introduce deformations of the scanner magnetic field.

Fiber-optic based systems typically move the LCD panels and their electronics away from the patient's eyes, to a separate unit outside the scanner bore but inside the magnet room. Fiber optic binocular glasses sit atop the patient's eyes and allow the patient to see the images created by the LCD panels. The images are carried by two separate bundles of fiber optic cables, one per LCD panel, to the binocular glasses. The fiber optic binocular glasses are made of plastic and they are safe. Yet, they still occupy space in the already crowded environment of the head coil with a patient's head. Also, the bundles of fiber optic cables that sit between the LCD panels and the binocular glasses are delicate and damage to even a single fiber may result in permanent loss of part of the image from one of the LCD displays.

Accordingly, there is a need for a method and apparatus for providing video to a patient in or near the bore of an MRI scanner where the provision of the video does not negatively impact the video does not negatively impact the MRI equipment and/or result of the MRI scan. There is also a need for a method and apparatus for providing high quality video to a patent inside the new smaller diameter, high performance, high resolution head coils now used for functional imaging.

### SUMMARY OF THE INVENTION

The subject invention relates to a method and apparatus for use with magnetic resonance imaging (MRI) and, in a specific embodiment, functional MRI imaging (fMRI). The subject invention can incorporate hardware and software to provide visual and/or auditory stimulation to a patient (or a volunteer) inside of an MRI scanner. The subject invention can also provide a response unit to collect responses from the patient. In an embodiment, the response unit can provide buttons for the patient to push in order to provide responses, including responses to visual and/or auditory stimulus provided to the patient. Synchronization of the visual and/or auditory stimulation provided to the patient with the scanner operations can be accomplished by utilizing, for example, an RF detector or TTL level pulses from the MR scanner. The subject invention can also include software tools to develop new functional MRI (fRI) experiment paradigms and a software package for fMRI data analysis.

### BRIEF DESCRIPTION OF DRAWINGS

FIG. 1 shows a schematic illustration of a patient within the bore of a MRI scanner, where a head coil is positioned around the patient's head and a specific embodiment of a patient display hood (PDH) in accordance with the subject invention is positioned in the MRI scanner so as to provide visual stimulus to the patient.

FIG. 2A shows a schematic layout of a MRI system, having a MRI scanner located in a magnet room and a control room adjacent to the magnet room, utilizing a prior art apparatus to provide video to a patient.

FIG. 2B shows a schematic layout of a MRI system, having a MRI scanner located in a magnet room and a control room adjacent to the magnet room, utilizing an embodiment of the subject invention to provide video to a patient.

FIGS. 3A-3C show a cross-sectional view of an embodiment of the subject PDH in accordance with the subject invention, where FIG. 3A shows an LCD panel and electronics enclosed in mesh and the PDH body prior to insertion of the LCD panel into the body of the PDH, FIG. 3B shows the PDH after insertion of the LCD panel and electronics enclosed in mesh, and FIG. 3C shows the PDH positioned relative to a head coil and patient and shows the light path from the LCD panel to the patients eyes.

FIG. 4 shows a block diagram of an embodiment of the subject invention.

FIG. 5 shows a console wiring diagram in accordance with an embodiment of the subject invention.

FIG. 6A shows a first layer and orientation of a mesh for enclosing the LCD panel and electronics of an embodiment of the subject PDH in accordance with the subject invention.

FIG. 6B shows a second layer and orientation of a mesh for enclosing the LCD panel and electronics of the embodiment of the subject PDH in accordance with the subject invention as shown in FIG. 6A.

FIG. 6C shows a side view of a fully assembled electronics enclosure with mesh, in accordance with the subject invention as shown in FIGS. 6A and 6B.

FIG. 7 shows a power distribution diagram for a PDH in accordance with the subject invention.

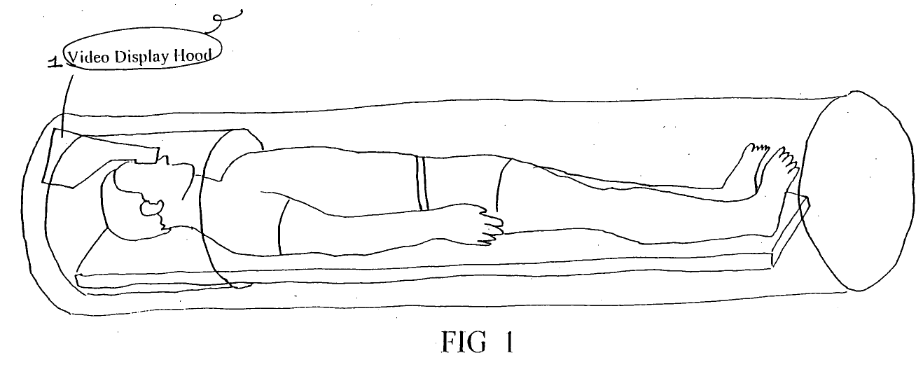

### DETAILED DISCLOSURE

The subject invention pertains to a method and apparatus for providing a video display near a MRI scanner. The subject invention can provide a visual display inside a magnet room in which a MRI scanner is located. In an embodiment, the subject invention can provide a visual display near or inside the bore of a MRI scanner so as to provide visual stimulation to a patient located in the bore of the MRI scanner. The visual display of the subject invention does not interfere with the operation of the MRI scanner and the MRI scanner does not interfere with the operation of the subject visual display. In a specific embodiment, the subject invention relates to a patient display hood that is transparent to the MRI scanner such that the magnetic fields of the MRI scanner do not significantly impact the operations of the subject PDH and such that the subject PDH produces minimal, if any, detectable RF noise in the MRI scanner.

In order to provide visual and/or auditory stimulation to a patient inside of an MRI scanner, some hardware is positioned in the magnet room. In a specific embodiment, a patient display hood (PDH) is positioned inside the MRI scanner. In an embodiment, the PDH can include a video display, such as a 15 inch video display. Other size video displays can also be utilized. In additional embodiments, the PDH can incorporate headphones to provide auditory stimuli. A response unit can be provided to the patient to allow the patient to provide feedback in response to the stimulus. In an embodiment of the subject invention, an optical fiber can carry the signals from the response unit, which is inside the MRI scanner, to outside of the MRI scanner, and vice versa if desired. In another embodiment, the optical fiber can carry the signals from the response unit to the PDH where the signals can be processed and another optical fiber can carry signals from the PDH to outside the MRI scanner. An RF detector can be incorporated with the PDH to allow synchronization with the MRI scanner. The subject PDH can receive power from a wall-mounted power supply through, for example, a bundle of copper-cable wires. In an embodiment, the subject PDH can connect to the control room hardware through a single fiber optic link.

The subject PDH unit can be designed to sit on top of the head coil of an MRI scanner. An opening can be provided on top of the head coil to provide access to the patient's eyes for the display of video. The PDH unit can be located in other positions as well, such that access to the patient's eyes is allowed. The patient can listen to audio through the headphones and can optionally speak to the operator using a microphone, which can be included with the subject PDH. In a specific embodiment, the response unit can be a keyboard-style unit including 10 buttons, one per digit (fingers and thumbs), to respond to the experimental paradigm and/or provide other feedback.

In an embodiment, the subject PDH can house the electronics positioned in the magnet room and is capable of functioning in the bore of the magnet while the MRI system is in operation. The functions of the PDH electronics can include, for example, one or more of the following: (1) receive signals from the control room via a fiber optic cable; (2) convert video signals from optical signals to digital electrical signals, for example in digital visual interface (DVI) format, where DVI is an interface standard for transmission of digital signals; (3) drive a liquid crystal display (LCD) screen; (4) power an LED based backlight for the LCD screen; (5) convert sound signals from optical signals to analog electrical signals; (6) power and drive the headphone unit; (7) drive the fiber-optic based button response unit; (8) encode the button presses from the patient onto the button response unit into a digital serial format and then to optical format to be sent to the control room; (9) detect the scanner RF pulse activity and convert this detected signal to optical format to be sent to the control room; (10) digitize the sound signal stream from the patient microphone and convert the sound signal to optical signals to be sent to the control room; and (11) control the operation of other serial devices such as game controllers.

The electronics incorporated into the subject PDH can preferably function without interference from the MRI scanner operations, including: the static magnetic field of the scanner, the dynamically changing magnetic fields (gradients) of the scanner, and the RF activity of the scanner, such that the magnetic fields of the MRI scanner do not significantly impact the operations of the subject system. In an embodiment, the subject PDH is transparent to the scanner operations. In an embodiment, the subject PDH is designed to minimize any possible deformations to the MRI scanner magnetic field and to produce minimal, if any, detectable RF noise. In an embodiment, the subject invention can provide video stimulation to a patient located in the isocenter region of an MRI scanner bore, which is the region of the MRI scanner having the most linear and most accurate area for MRI scanning.

The subject PDH is also designed for the safety of the patient and operator. In a specific embodiment, the patient is not in contact with any electrical component that, in case of failure, could create the potential for a dangerous situation.

The operator can manage the functions of the subject PDH from a console located outside of the magnet room. The console can be located in the control room of the MRI scanner. Preferably, the console is located close to the MRI scanner console. In an embodiment, the console can include: a computer rack, a large screen display, a camera, a microphone, speakers, a keyboard, and a mouse. The computer rack can include a master control unit (MCU) and two computers. The first of the two computers, which can be referred to as the experiment presentation computer (EPC), can generate stimuli (auditory and/or visual) that are sent to the patient through the MCU. In a specific embodiment, the EPC can include 1.8 GHz Pentium 4 computer with Windows XP Professional, 512 MB RAM, 120 GB hard drive, SVGA graphics card, DVD/CD drive, Sound Blaster Audio PCI 16 sound card, 10/100/1000 BaseT network card, and removable hard drive. The second computer, which can be referred to as the control and analysis computer (CAC), can control operations of the subject system. In a specific embodiment, the CAC can include 2.0 GHz Xeon computer with Windows XP Professional, 1 GB RAM, 120 GB hard-drive, DVD/RW Drive, graphics card with GForce 4 chipset, Sound Blaster Audio PCI 16 sound card, 10/100/1000 BaseT network card, and removable hard drive. The master control unit can serve one or more of the following tasks: (1) route sound signals and video signals from the EPC (or other sources) to the PDH over a fiber optic cable; (2) receive button response signals from the PDH and route them to the EPC; (3) receive sound signals from the patient microphone and route them to the operator speakers; (4) route keyboard and mouse signals to the appropriate computer as selected by the operator; (5) receive synchronization signals from the PDH and route them to the EPC; and (6) route the sound and video signals of the EPC to a desktop operator monitor.

The subject invention, in various embodiments, can provide one or more of the following advantages: (1) the entire PDH, the only component of the subject system installed in the Magnet Room apart from the wall-mounted power supply, can be introduced inside of the bore of the MRI scanner; (2) the subject PDH can incorporate electronics that can operate reliably inside of the MRI scanner and that are transparent to scanner operations; (3) the subject PDH can incorporate a high resolution (1024×768 pixels), large (15″ inches) LCD color screen; (4) the subject display can cover approximately 30° of patient's field of view, or visual field, in the position of the patient (which corresponds to approximately 80% of the visual cortex in the occipital areas of the human brain); (5) the entire electronics that controls the LCD screen can be housed within the PDH; (6) the LCD backlight and its powering circuit is adapted to function inside of the PDH without interfering with the operations of the MRI scanner; (7) the patient response unit can utilize a plurality of buttons and can incorporate fiber-optic switches, with the electronics enclosed within the subject PDH; (8) the PDH can incorporate electronics to drive additional serial devices, such as game controllers.

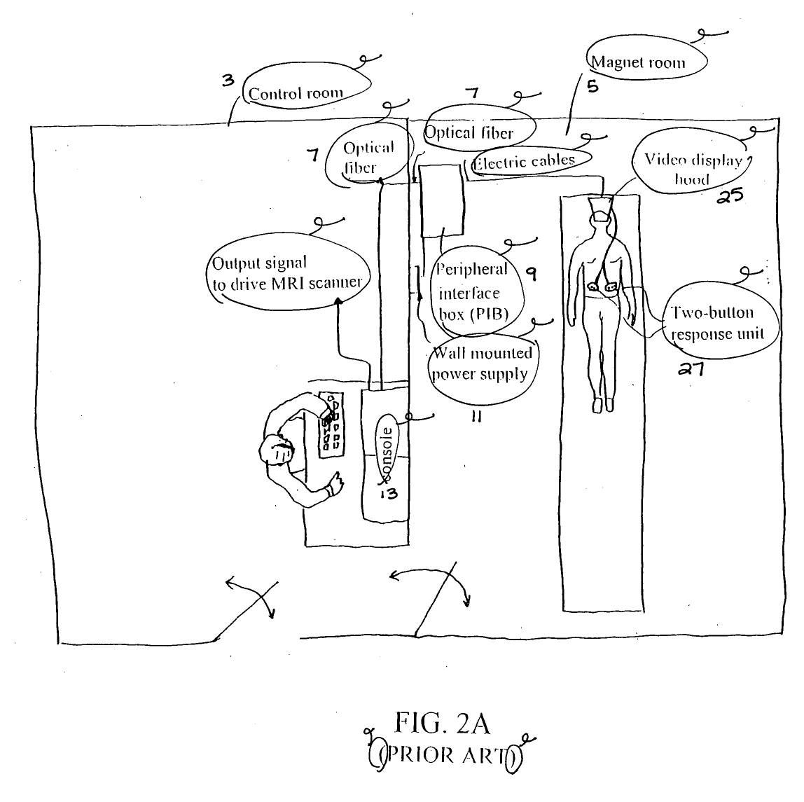

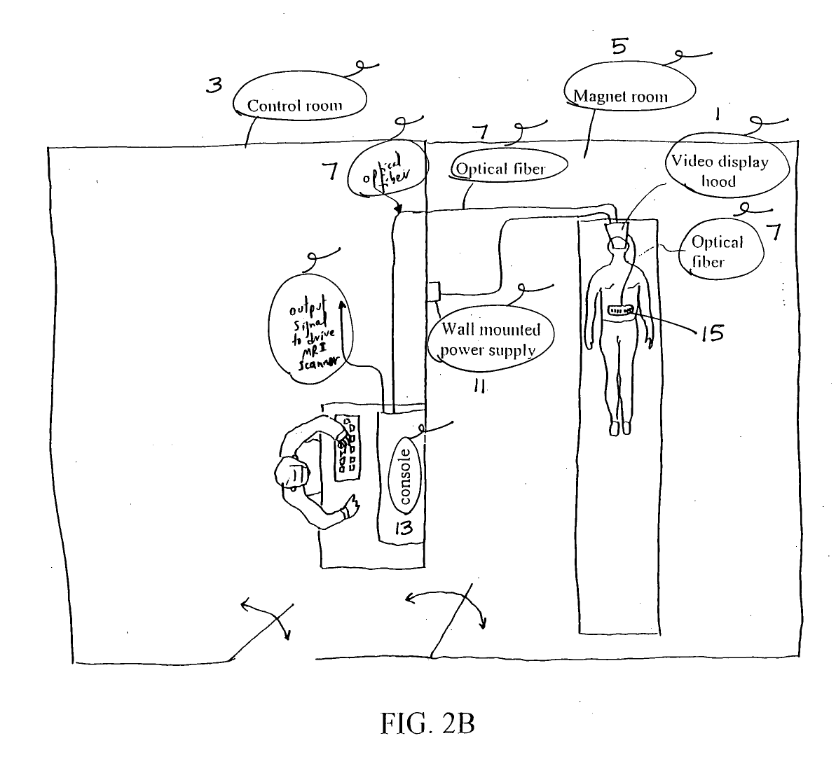

Referring to FIG. 2B, a MRI system, having a MRI scanner located in a magnet room 5 and a control room 3 adjacent to the magnet room 5, utilizing an embodiment of the subject invention for providing video to a patient, is shown. In this embodiment, the electronics needed to interact with the various components associated with the PDH 1 are located within the PDH 1. Such electronics include electronics for interfacing with at least one or more of the following: LCD screen, the headphones, the patient response unit 15, and the RF antennae for detecting MRI scanner operation. In contrast, with the system shown in FIG. 2A where the electronics in the magnet room 5 are split in two main units (PIB 9 and Video Display Unit), the subject PDH 1 can house the electronics for the video display and PIB 9 functions. Additionally, the PIB 9 of the system shown in FIG. 2A is designed to work inside the magnet room 5, but outside the MRI scanner bore. In contrast, in accordance with an embodiment of the subject invention, the entire PDH 1 of the subject invention can work inside the MRI scanner bore.

In an embodiment, the subject system features a 15″ video that provides a 30-degree visual field of view (FOV). Preferably, the FOV is at least 30-degrees right-to-left and 30-degrees up and down. In contrast, the system shown in FIG. 2A provides a 7″ video, with a 15-degree FOV. The increased FOV of the subject system provides a significant advantage to fMRI studies that rely on the coverage of visual areas. It also gives a more natural coverage of the visual field for alternative uses of the system, e.g. patient entertainment.

The resolution of the screen in the system shown in FIG. 2A is 640×480 pixels. In an embodiment, the resolution of the screen in the system shown in FIG. 2B is 1024×768 (1280×1024 max). The system shown in FIG. 2A uses analog VGA signals such that during the assembly stage, each systems has to be carefully calibrated for optimal matching of the MCU (sender), fiber optic cable 7, and PIB 9 (receiver). In an embodiment, the system shown in FIG. 2B can use digital DVI signals to drive the video such that due to the digital nature of the signals that are transmitted through the optical media, there is no need for calibration for optimal matching of the MCU (sender), fiber optic cable 7, and PEB 9 (receiver).

The subject invention can incorporate a BRU 15 having a one piece boomerang shape with 10-buttons. The 8 buttons on the top of the BRU 15 can allow the patient's fingers to provide patient response. The 8 buttons can resemble a piano key layout. The buttons for the patient's thumbs can be positioned on the bottom of the BRU 15, allowing the patient to easily maneuver the BRU 15 during use. This BRU 15 can incorporate an ergonomic design that allows it to be used by a larger segment of the population. The subject BRU 15 can connect to the PDH 1 via one or more fiber optic cables 7 and the subject PDH 1 can incorporate electronics to decode the optic signals, encode them into a serial data stream, and send them through the fiber optic cable(s) 7 to the console 13.

When compared to projector-based systems, the subject system can have one or more of the following advantages:

1. The subject system can provide a complete turnkey solution for fMRI. The subject system includes all the hardware and software for fMRI sessions with visual and/or auditory stimuli and can allow the collection of responses from the patient. Synchronization with the scanner can be accomplished by utilizing RF pulses or TTL signals. In contrast, projector-based systems often require some sort of custom electronics to synchronize operations with the scanner.

2. The subject system can reduce setup time. In an embodiment, setup includes positioning the PDH onto the top of the head coil. In contrast, a projector system can require the positioning of the LCD projector, projection screen, head coil mirror, and, critically, the position of the screen must be adjusted to find the best focus of the image on the screen. This setup time for projector systems is spent setting up the apparatus for the visual stimuli only—more setup time can be needed if the projector is integrated with audio and button response boxes.

3. The subject screen can cover a greater FOV. The subject display can cover a 30 degree FOV, independent of the shape and size of the scanner, the size of magnet room, and patient's body size.

4. The images on the subject video screen do not suffer from the optical diffraction of the projector optics.

5. The subject system is transparent of operations with the scanner.

The subject system may enjoy one or more of the following advantages when compared to goggle-based systems:

1. In an embodiment, the subject system provides an fMRI turnkey solution that includes hardware for visual and/or auditory stimulation, collection of patient responses, and synchronization with the MRI scanner, plus software for control and delivery of fMRI experimental paradigms and fMRI data analysis. In contrast, goggle-based systems typically only include the hardware for visual presentation and, in some cases, the software to deliver the experimental paradigm.

2. The subject PDH can sit on top of the head coil and, in an embodiment, can leave the whole space inside of the head coil available for the patient. This can be important with the current generation of high performance head coils, which are designed to have a tight fit to the head of the patient and leave little or no room to introduce external hardware inside of the coil.

3. The subject PDH can sit close to the patient head, but not exactly on top of the eyes as in the case of the LCD goggles, thus reducing concerns about the safety of the system. The subject PDH can be utilized with the HRH head coil from MRI Devices Corporation.

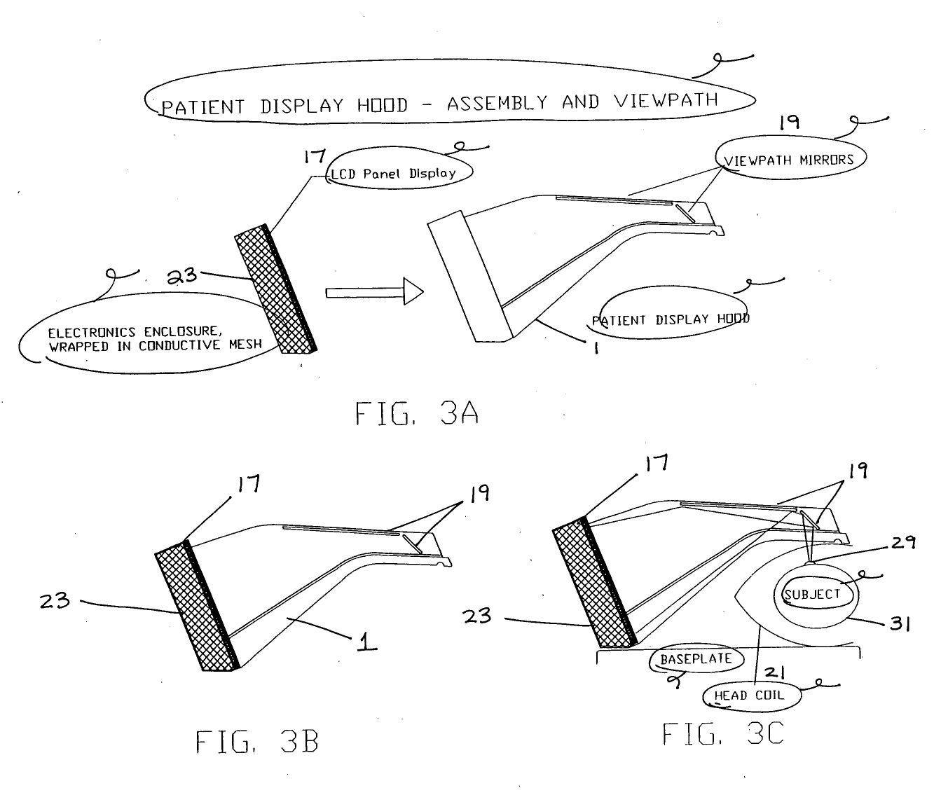

A specific embodiment of the subject PDH is shown in FIGS. 3A-3C. The PDH 1 houses a LCD display 17 and associated electronics. The LCD display panel 17 and other electronics can be surrounded by mesh 23 so that the LCD display panel 17 and electronics do not interfere with the MRI scanner and the MRI scanner does not interfere with the LCD display panel 17 and other electronics. As shown in FIG. 2B, control signals can be received from a console 13 in the control room 3 through one or more optical fibers 7, while power to drive the PDH components can be received from a wall mounted power supply 11 in the magnet room 5 via one or more copper wire bundles. Other materials, such as aluminum wire bundles, can be used to carry electrical power to the PDH 1. The PDH 1 shown in FIGS. 3A-3C has an outer housing with RF shielding material inside the housing to shield the electronics within the PDH 1 from fields produced by the MRI scanner and to shield fields produced by the PDH electronics from interfering with the MRI scanner. The PDH shown in FIGS. 3A-3C also includes mirrors 19 to guide the image produced by the LCD screen 17 to the patient's eyes 29. These mirrors 19 can allow the use of a larger LCD display panel 17 in the crowded bore of the MRI scanner, with the patient's head 31 and the head coil 21 taking up much space.

The subject invention also relates to a head coil—PDH combination. In an embodiment a high resolution head coil 21 (HRH head coil) from MRI Devices Corporation can be combined with the subject PDH to form a head coil—PDH combination.

The subject system can incorporate an LCD display. The subject invention also relates to an LCD display. The subject display can be incorporated into a video display system in accordance with the subject invention. In an embodiment, the subject LCD display is modified compared to standard LCD displays, in order to operate in the subject PDH within the MRI scanner bore. Standard LCD displays come with cold cathode fluorescent tubes (CCFT), which are basically fluorescent bulbs that cannot provide stable light within the MRI scanner bore. The subject LCD display can incorporate

LED's to provide the light that the CCFT's would have provided. In order to provide enough light from LED's, typical circuitry would create much heat. In an embodiment, the subject system incorporates circuitry to power the LED's for the LCD display that reduces power consumption within the subject PDH by supplying the required voltage from an external power supply located out of the MRI scanner bore. Advantageously, positioning the power supply used to drive the LCD external to the PDH located inside the MRI scanner bore can allow the removal of ferrous components from the LCD power circuitry located inside the MRI scanner and can reduce RF noise from the PDH located within the scanner.

In an embodiment, the subject system delivers digitized signals via fiber optic cables directly to the drive electronics in the PDH. This can permit delivery of higher quality audio and video signals to the patient than achievable by analog electronic signals delivered into the magnet bore.

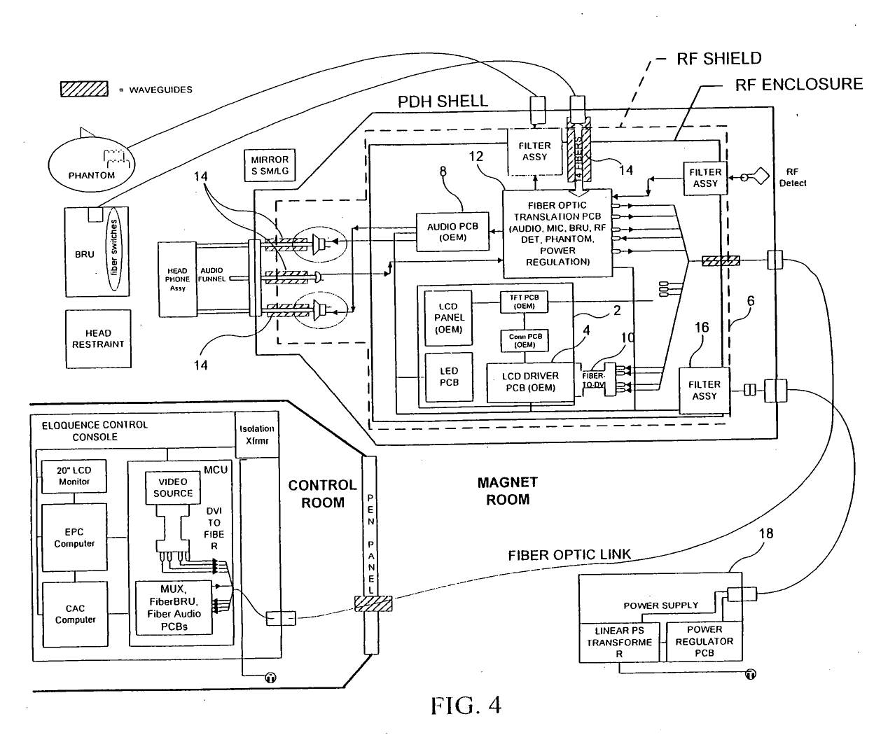

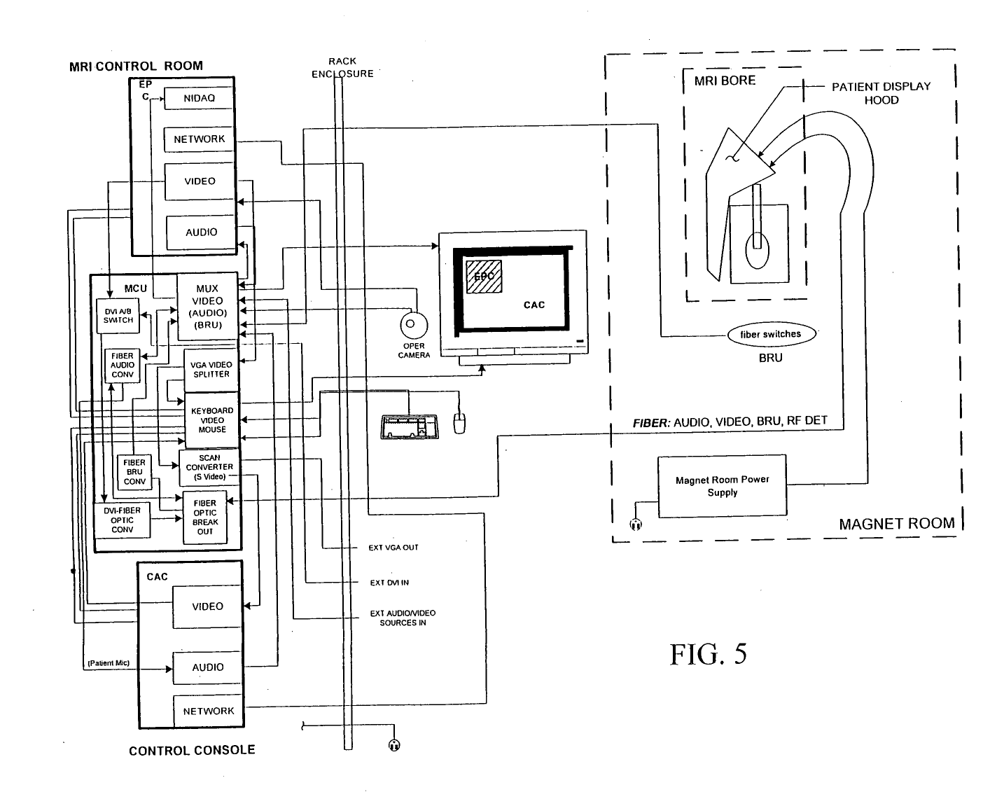

In an embodiment, the subject invention's electronic assemblies housed in the PDH can function in the MRI magnet bore without interference from or causing interference to, the MRI scanner operations. Referring to FIG. 4, a specific embodiment of the subject invention is shown, incorporating an LCD and electronic control circuitry that does not interfere with, and operates without interference from, the MRI scanner inside of which it is placed. In a specific embodiment, the LCD is arrived at by modifying a LCD based on typical design criteria.

In a specific embodiment, the metallic LCD housing, the LCD driver and interface printed circuit boards (PCBs), the power inverter PCB for the cold cathode fluorescent tubes (CCFT) that provide back lighting, the CCFTs themselves, and the DC converter PCB providing the various DC voltages to all of the LCD circuitry of such a typical LCD can be modified to allow functioning in the MRI scanner in accordance with the subject invention.

The ferrous metal LCD housing is removed and replaced with a non-metallic housing 2. The non-metallic housing is designed to securely capture the LCD, it's active matrix PCBs, video filter layers, and backlight sources. This housing also incorporates a means for mounting the LCD controller, driver, and interface PCBs, as well as the PDH interface electronics PCB.

The ferrous filtering components associated with an on-board DC regulator, and the regulator itself, of the LCD driver PCB 4 are removed from the LCD driver PCBs. The voltage that was to be supplied by the removed regulator is then supplied by an external source. Ferrous shields on video input connectors, along with any other unused ferrous connectors are also removed. The DC converter PCB is removed from the interface PCB along with other ferrous filtering components. The various voltages to be supplied to the LCD by this modified PCB is then supplied from an external source.

The power inverter PCB used to generate the high AC voltage for the CCFTs under normal operation, and the CCFTs themselves, are removed. An alternate back lighting technology is utilized in place of the CCFTs. In a specific embodiment, white LEDs are used for the backlighting. The voltage to power the white LEDs is supplied from an external source.

Once all of the described modifications have been completed, the LCD and all the above components are reassembled into and mounted onto the substituted non-metallic housing.

In alternative embodiments, and LCD in accordance with the subject invention can be made without modifying a typical LCD.

The subject invention can also utilize a fiber optic-to-DVI video converter 10. Again, a typical fiber optic-to-DVI video converter can be modified to allow its placement in the MRI scanner, or a fiber optic-to-DVI video converter can be made for use in the subject invention. In modifying a typical fiber optic-to-DVI video converter, ferrous hardware and connector shields are removed and such hardware is replaced with non-ferrous hardware.

An audio amplifier PCB 8 can be removed from its housing and ferrous filter and power inverter components can be removed. The multiple voltages used by the amplifier can then be supplied from an external source. Other components can be removed to control voltage distribution on the PCB. Electrostatic speakers can be used as they have a non-ferrous construction.

The subject invention can also incorporate a PDH interface electronic PCB 12, which controls one or more of the following: audio, video, data interface, and fiber-to-electronic signal conversion in the PDH. Such a PDH interface electronic PCB can be designed to minimize ferrous components and eddy currents induced by magnetic fields and RF gradients generated by the MRI system.

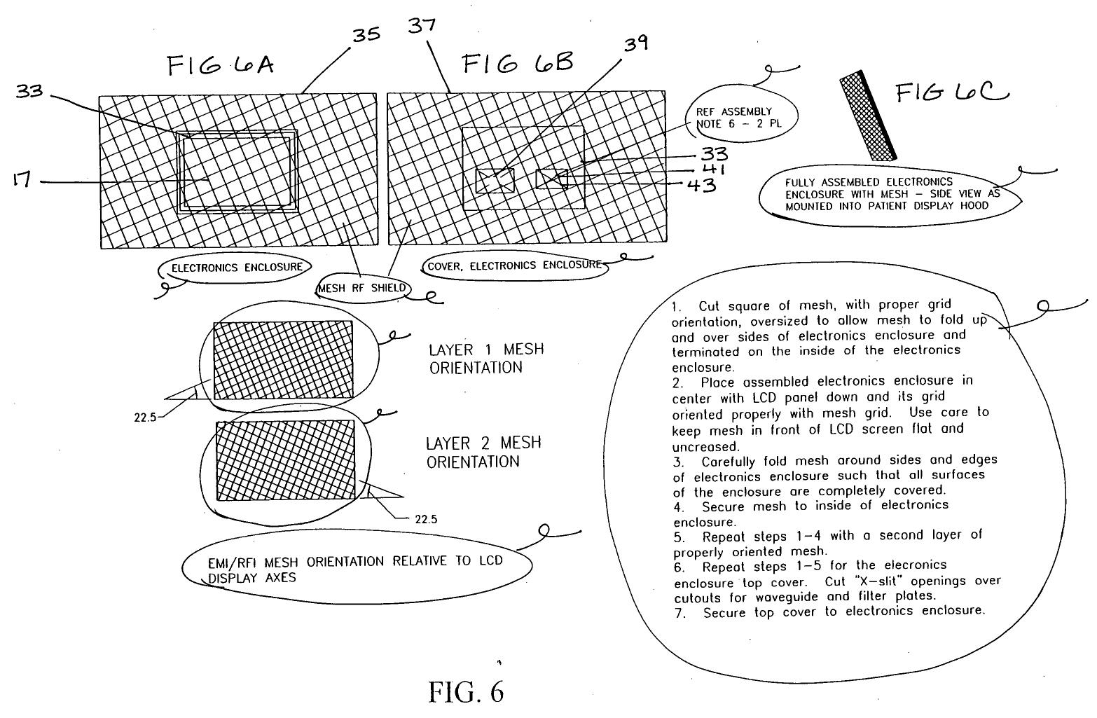

Referring to FIGS. 6A-6C, shielding is shown that can be utilized with the subject invention such that electronics in the subject PDH can be shielded to minimize, or prevent, RF noise generated by the electronics from interfering with image generation by the MRI system and to prevent RF fields from the MRI scan operation from interfering with the functionality of the electronics. In an embodiment, shielding of the LCD display panel and other electronics in the subject PDH can be accomplished by housing the electronics in a Faraday enclosure. This shielding can be accomplished by surrounding the LCD display panel and other electronics with a conductive mesh. Referring to FIGS. 6A-6C, steps for a mesh wrapping procedure in accordance with an embodiment of the subject invention is provided as follows.

1) Cut square of mesh 35, with proper grid orientation, oversized to allow mesh 35 to fold up and over sides of electronics enclosure 33 and terminated on the inside of the electronics enclosure 33.

2) Place assembled electronics enclosure 33 in center with LCD panel 17 down and its grid oriented properly with mesh grid. Use care to keep mesh 35 in front of LCD screen flat 17 and uncreased.

3) Carefully fold mesh 35 around sides and edges of electronics enclosure 33 such that all surfaces of the enclosure are completely covered.

4) Secure mesh 35 to inside of electronics enclosure 33.

5) Repeat steps 1-4 with a second layer of properly oriented mesh 37.

6) Repeat steps 1-5 for the electronics enclosure 33 top cover. Cut “X-slit” openings 43 over cutouts for waveguide 41 and filter plates 39.

7) Secure top cover to electronics enclosure 33.

A fully assembled enclosure is shown in 6C as it can be mounted into the patient display hood. The Faraday enclosure itself should be, preferably, immune to eddy currents generated by the MRI magnet gradients. FIG. 3A shows an embodiment where a mesh is placed around the LCD display panel 17 and electronics and the mesh is selected such that the patient can see through the mesh in order to view the LCD display. In a specific embodiment, the shielding can be essentially transparent to the patient in front of the LCD display panel 17 to allow the patient an essentially unimpeded view of the LCD display panel 17. In a specific embodiment, this can be achieved by wrapping the LCD display panel 17 in a conductive mesh, whose transparency and physical construction parameters, along with assembly orientation relative to the LCD pixel grid are carefully selected to simultaneously shield properly and allow the patient to see the LCD display. The parameters that can impact these requirements include the shielding material, diameter of the fibers in the shielding mesh, spacing between the fibers, and orientation of the mesh fibers relative to the LCD display pixel grid parameters.

The technique used for wrapping the enclosure in shielding mesh is important to ensure a true Faraday RF shield is achieved. In a specific embodiment, the subject system incorporates two layers of 0.0016″ dia., 250 opi phosphor-bronze mesh, whose layers, in order to eliminate Moire patterns and visibility of the mesh, are oriented such that the mesh grid of the two layers are oriented at 45 degrees relative to each other. In a further specific embodiment, referring to FIGS. 6A-6C, the two mesh grids 35 and 37 are each offset by 22.5 degrees relative to the LCD grid such that the two mesh grids 35 and 37 are oriented at 45 degrees relative to each other. There can be more than one set of mesh parameters that can meet the transparency and conductive requirements. Optimized parameters may vary with LCD pixel and grid design as well. The orientation angles used by the subject system can vary depending on the LCD pixel grid design.

Because the best mesh parameters for transparency diverge from the best mesh parameters for conductivity (and cost), an alternate PDH RF Faraday shield 6 design can incorporate an “RF window” in front of the LCD screen. This RF window can incorporate superior optical transparency parameters, captured in a clear laminate with the mesh exposed at the perimeter in order to mate with a mesh having better (and less expensive) conductive parameters, which is used for enclosing the body of the RF electronics enclosure. A specific design that can be incorporated with the subject system uses a stainless steel 0.001″ diameter, 230 opi mesh, with blackened silver plated fused wire crossovers. Other similar meshes can also be used with an embodiment incorporating an RF window.

Pneumatic audio signals, generated from electrostatic speakers in the subject RF Faraday enclosure, can be transferred out of the RF Faraday enclosure 6 through plastic tubing that can penetrate the RF Faraday enclosure through waveguides 14. Fiber optic cable can also penetrate the RF Faraday enclosure 6, without compromising its shielding, by passing through waveguides 14 built into the enclosure housing.

A Faraday shielded low-pass filter assembly 16 can be used to condition all external voltages penetrating the RF Faraday 6 from the external power supply 18. An LC low-pass filter can be used on each of the power conductors from the external supply.

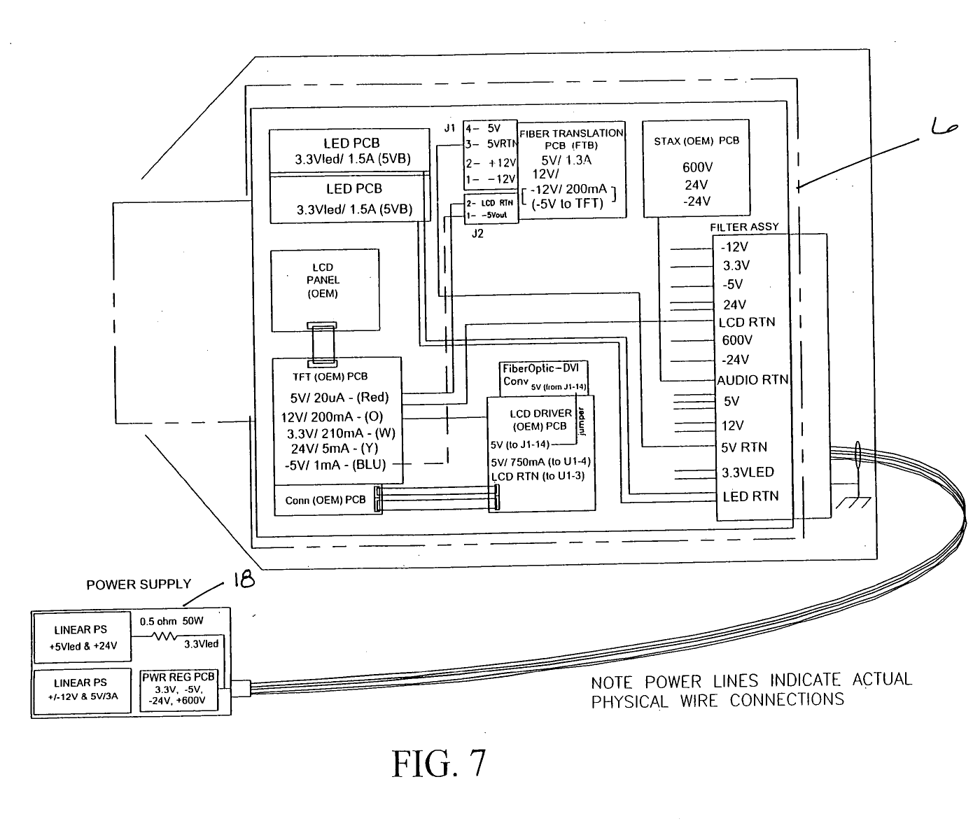

Referring to FIG. 7, the subject invention can utilize externally supplied voltages to power the electronics within the RF Faraday enclosure 6. Such externally supplied voltages can be supplied by a multiple output linear DC power supply mounted in the magnet room and outside the magnet bore. A linear supply can be used to minimize potential ambient room electronic noise resulting from power supply conversion circuitry. The power supply should be securely mounted as it incorporates ferrous material. Careful attention should be paid to the physical distribution wires to preserve the functionality of the above circuits in the magnet bore.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

1. A method for providing a visual display inside a magnetic resonance imaging magnet room, comprising:
locating a patient display hood in a magnetic resonance imaging magnet room, wherein the patient display hood comprises:
a means for receiving video signals;
a liquid crystal display (LCD) panel;
an LCD driver to drive the LCD panel;
a means to input the received video signals to the LCD panel;
an LED based backlight for the LCD panel;
circuitry to power the LED based backlight for the LCD panel; and
shielding to shield the following:
the means for receiving video signals;
the LCD panel;
the LCD driver;
the means to input the received video signals to the LCD panel;
the LED based backlight for the LCD panel; and
the circuitry to power the LED based backlight for the LCD panel;
inputting video signals to the means for receiving video signals; and
displaying the received video signals on the LCD panel so as to provide a visual display, wherein the patient display hood is substantially transparent to a MRI scanner such that the magnetic fields of the MRI scanner do not significantly impact the operations of the patient display hood and such that the patient display hood produces negligible detectable RF noise in a MRI scanner.

2. The method according to claim 1, wherein the patient display hood comprises at least one mirror for directing the visual display on the LCD panel to a patient, further comprising:
providing video stimulation to a patient located in the isocenter region of a MRI scanner in the magnetic imaging magnet room, wherein providing video stimulation to a patient located in the isocenter region of the MRI scanner comprises directing the visual display on the LCD panel to the patient via the at least one mirror.

3. The method according to claim 1, wherein receiving video signals comprises receiving optical video signals via a fiber optic link.

4. The method according to claim 3, further comprising:
converting the received optical video signals to digital electrical signals.

5. The method according to claim 1, wherein locating a patient display hood in a magnetic resonance imaging magnet room comprises locating the patient display hood in a bore of a MRI scanner in the magnetic imaging magnet room.

6. The method according to claim 2, wherein providing visual stimulation to a patient located in the isocenter region of the MRI scanner comprises providing a visual field of view to the patient, wherein the visual field of view is at least 30-degrees right-to-left and 30-degrees up and down.

7. The method according to claim 5, further comprising:
synchronizing displaying the received video signals on the LCD panel with the MRI scanner.

8. The method according to claim 7, wherein synchronizing displaying the received video signals on the LCD panel with the MRI scanner comprises detecting RF pulses from the MRI scanner.

9. The method according to claim 1, further comprising locating a power supply to provide power to the circuitry to power the LED based backlight for the LCD panel out of the MRI scanner bore.

10. The method according to claim 1, wherein the shielding comprises a Faraday enclosure.

11. The method according to claim 1, wherein the shielding comprises a conductive mesh.

12. The method according to claim 2, wherein the shielding comprises a conductive mesh, wherein the patient can see through the conductive mesh to view the visual display on the LCD panel.

13. The method according to claim 12, wherein the conductive mesh is essentially transparent to the patient.

14. The method according to claim 13, wherein the shielding comprises two layers of conductive mesh, wherein the mesh grid of the two layers of conductive mesh are oriented at about 45 degrees relative to each other.

15. The method according to claim 14, wherein the mesh grids of the two layers are each offset by about 22.5 degrees relative to the LCD panel grid.

16. The method according to claim 2, further comprising:
providing a response unit to the patient, wherein the response unit allows the patient to provide feedback.

17. The method according to claim 2, further comprising:
providing auditory stimulation to the patient.

18. The method according to claim 1, further comprising:
locating a console outside of the magnetic resonance imaging magnet room, wherein the console allows an operator to control the patient display hood.

19. The method according to claim 18, further comprising:
linking the console and the patient display hood via one or more fiber optic cables.

20. An apparatus for providing a visual display inside a magnetic resonance imaging magnet room, comprising:
a patient display hood, wherein the patient display hood comprises:
a means for receiving video signals;
a liquid crystal display (LCD) panel;
an LCD driver to drive the LCD panel;
a means to input the received video signals to the LCD panel;
an LED based backlight for the LCD panel;
circuitry to power the LED based backlight for the LCD panel; and
shielding to shield the following:
the means for receiving video signals;
the LCD panel;
the LCD driver;
the means to input the received video signals to the LCD panel;
the LED based backlight for the LCD panel; and
the circuitry to power the LED based backlight for the LCD panel, a
means for inputting video signals to the means for receiving video signals;
wherein the received video signals are displayed on the LCD panel so as to provide a visual display, wherein the patient display hood is substantially transparent to a MRI scanner such that the magnetic fields of the MRI scanner do not significantly impact the operations of the patient display hood and such that the patient display hood produces negligible detectable RF noise in a MRI scanner.

21. The apparatus according to claim 20, wherein the patient display hood comprises at least one mirror for directing the visual display on the LCD panel to a patient.

22. The apparatus according to claim 20, further comprising a fiber optic link for transmitting optical video signals from the means for inputting video signals to the means for receiving video signals to the means for receiving video signals.

23. The apparatus according to claim 22, further comprising:
a means for converting received optical video signals to digital electrical signals.

24. The apparatus according to claim 20, wherein the patient display hood is substantially transparent to a MRI scanner when the patient display hood is located in a bore of a MRI scanner in the magnetic imaging magnet room.

25. The apparatus according to claim 21, wherein the patient display hood provides a visual field of view to the patient, wherein the visual field of view is at least 30-degrees right-to-left and 30-degrees up and down.

26. The apparatus according to claim 24, further comprising:
a means for synchronizing the displayed received video signals on the LCD panel with the MRI scanner.

27. The apparatus according to claim 26, wherein the means for synchronizing the displayed received video signals on the LCD panel with the MRI scanner comprises a means for detecting RF pulses from the MRI scanner.

28. The apparatus according to claim 20, further comprising a power supply to provide power to the circuitry to power the LED based backlight for the LCD panel located out of the MRI scanner bore.

29. The apparatus according to claim 20, wherein the shielding comprises a Faraday enclosure.

30. The apparatus according to claim 20, wherein the shielding comprises a conductive mesh.

31. The apparatus according to claim 21, wherein the shielding comprises a conductive mesh, wherein the patient can see through the conductive mesh to view the visual display on the LCD panel.

32. The apparatus according to claim 31, wherein the conductive mesh is essentially transparent to the patient.

33. The apparatus according to claim 32, wherein the shielding comprises two layers of conductive mesh, wherein the mesh grid of the two layers of conductive mesh are oriented at about 45 degrees relative to each other.

34. The apparatus according to claim 33, wherein the mesh grids of the two layers are each offset by about 22.5 degrees relative to the LCD panel grid.

35. The apparatus according to claim 21, further comprising: a response unit, wherein the response unit allows the patient to provide feedback.

36. The apparatus according to claim 21, further comprising:
a means for providing auditory stimulation to the patient.

37. The apparatus according to claim 20, further comprising:
a console located outside of the magnetic resonance imaging magnet room, wherein the console allows an operator to control the patient display hood.

38. The apparatus according to claim 37, further comprising:
one or more fiber optic cables linking the console and the patient display hood.
